(19) United States
(12) Patent Application Publication
(10) Pub. No.: US 2010/0143893 A1
(43) Pub. Date: Jun. 10, 2010

(54) Method for detection of cytosine methylation

(76) Inventor: Kurt Berlin, Stahnsdorf (DE)

Correspondence Address: Epigenomics AG, Kleine Praesidentenstr. 1, Berlin, 10178, DE

(21) Appl. No.: 10/557,320

(22) PCT Filed: May 21, 2004
(86) PCT No.: PCT/EP04/05553

§ 371 Date: Dec. 18, 2006

Related U.S. Application Data
(60) Provisional application No. 60/472,041, filed on May 20, 2003.

(51) Int. Cl. C12Q 1/68; C12P 19/34

(52) U.S. Cl. 435/6; 435/91.2

(57) ABSTRACT

Herein described is a method for the detection of cyto-sine methylation in a nucleic acid sample, comprising the steps of: a) treating a nucleic acid sample with an agent convert-ing unmethylated cytosine bases into uracil bases and not converting methylated cytosine bases within said nucleic acid sample, b) amplifying selected segments of the treated nucleic acid sample, by providing two first oligonucleotide primers (A and B) that are capable of producing an amplificate under certain chosen amplification conditions inde-pendently of the methylation status of the nucleic acid before treatment in step a), and further providing at least two additional second oligonucleotide primers (C and D) that can each produce a product with one of the first primers (A or B) under said same amplification conditions, wherein at least one of the second primers binds to the nucleic acid in a methylation specific manner, thereby distinguishing between unconverted initially methylated and converted unmethylated nucleic acids and/or blocking molecules are provided that hinder the binding of at least one of the second primers to the nucleic acid in a methylation specific manner, thereby distinguishing between unconverted initially methylated and converted unmethylated nucleic acids, c) detecting the amplificates of the treated nucleic acid.

### BACKGROUND

The present invention relates to detection of cytosine methylation in nucleic acid samples.

The development of methods in molecular biology in recent years has led to levels of observation including genes themselves, transcription and translation of these genes into RNA, and proteins arising from gene expression. The activation and inhibition of certain genes in certain cells and tissues during the course of development of an individual can be correlated with the extent and nature of the methylation of the genes or of the genome. Furthermore, pathogenic states are known to be expressed by a modified methylation pattern of individual genes or of the genome.

5-Methylcytosine is the most frequent covalently modified base in the DNA of eukaryotic cells, and plays a role in the regulation of transcription, in genetic imprinting, and in tumorigenesis. The identification of 5-methylcytosine as a component of genetic information is thus of considerable interest. However, 5-Methylcytosine positions cannot be identified by sequencing, since 5-methylcytosine has the same base-pairing behavior as cytosine. In addition, in the case of PCR amplification, epigenetic information, which is borne by 5-methylcytosines, is completely lost.

A number of methods for investigating DNA or other nucleic acid samples for the presence of 5-methylcytosine is based on the specific reaction of bisulfite with cytosine. The bisulfite reaction selectively converts cytosine—but not 5-methylcytosine—to uracil, which corresponds in its base-pairing behavior to thymine. After DNA treatment with bisulfite reaction, 5-methylcytosine can be detected by standard molecular biological techniques as the single remaining cytosine—for example, by amplification and hybridization or sequencing—whereas 5-methylcyto sine cannot be distinguished in an untreated DNA sample from cytosine by means of its hybridization behavior.

Prior art is directed at the sensitivity of the bisulfite reaction and includes a method that incorporates a DNA sample in an agarose matrix through which diffusion and renaturation of the DNA is prevented (bisulfite reacts only on single-stranded DNA) and precipitation and purification steps are replaced by rapid dialysis (Olek, A., Oswald, J., Walter, J., “A Modified and Improved Method for Bisulphite Based Cytosine Methylation Analysis,” Nucleic Acids Res., 1996, Dec. 15; 24 (24): 5064-6). Individual cells can be investigated with this method. Previously, only individual regions of DNA up to approximately 3000 base pairs in length had been investigated, and a global investigation of cells for thousands of possible methylation analyses had not been possible. However, this method cannot reliably analyze very small fragments of small sample quantities, which may be lost despite protection from diffusion through the matrix.

Other known techniques for detecting 5-methylcytosines can be derived from Rein, T., DePamphilis, M. L., Zorbas, H., “Identifying 5-Methylcytosine and Related Modifications in DNA Genomes,” in Nucleic Acids Res., 1998, May 15; 26 (10): 2255-64, but likewise do not allow analysis of small sample quantities of DNA.

The bisulfite technique has previously been applied only in research, with a few exceptions (e.g., Zeschnigk, M., Lich, C., Buiting, K., Dorfler, W., and Horsthemke, B., “A Single-Tube PCR Test for the Diagnosis of Angelman and Prader-Willi Syndrome Based on Allelic Methylation Differences at the SNRPN Locus,” Eur. J. Hum. Genet, 1997, Mar-Apr; 5 (2): 94-8). Short, specific pieces of a known gene are always amplified after a bisulfite treatment, followed by complete sequencing (Olek, A., Walter, J., “The Pre-Implantation Ontogeny of the H19 Methylation Imprint,” Nat. Genet., 1997, Nov.; 17(3): 275-6)) or by detection by a “primer extension reaction” of individual cytosine positions (Gonzalgo, M. L., Jones, P. A., “Rapid Quantification of Methylation Differences at Specific Sites Using Methylation-Sensitive Single Nucleotide Primer Extension (Ms-SNuPE),” Nucleic Acids Res., 1997, Jun. 15;25 (12):2529-31; WO 95/00669) or an enzyme cleavage (Xiong, Z, Laird, P. W., “COBRA: A Sensitive And Quantitative DNA Methylation Assay,” Nucleic Acids Res., 1997, Jun. 15;25(12): 2532-4). Detection by means of hybridizing has been described (Olek et al., WO 99/28498).

Urea improves the efficiency of the bisulfite treatment prior to the sequencing of 5-methylcytosine in genomic DNA (Paulin, R., Grigg, G W., Davey, M. W., Piper, A. A., “Urea Improves Efficiency of Bisulfite-Mediated Sequencing of 5′-Methylcytosine in Genomic DNA,” Nucleic Acids Res., 1998, Nov. 1;26(21):5009-10).

Other publications concerned with the application of the bisulfite technique for the detection of methylation in individual genes are: Grigg, G., Clark, S., “Sequencing 5-Methylcytosine Residues in Genomic DNA,” Bioassays, 1994, Jun.;16(6): 431-6, 431; Zeschnigk, M., Schmitz, B, Dittrich, B., Buiting, K., Horsthemke, B., Dorfler, W., “Imprinted Segments In The Human Genome: Different DNA Methylation Patterns In The Prader-Willi/Angelman Syndrome Region As Determined By The Genomic Sequencing Method,” Hum. Mol. Genet., 1997, Mar; 6(3):387-95; Feil, R., Charlton, J., Bird, A. P., Walter, J., Reik, W., “Methylation Analysis On Individual Chromosomes: Improved Protocol For Bisulfite Genomic Sequencing,” Nucleic Acids Res. 1994 Feb. 25;22(4): 695-6; Martin, V., Ribieras, S, Song-Wang, X, Rio, M. C., Dante, R., “Genomic Sequencing Indicates A Correlation Between DNA Hypomethylation In The 5′ Region Of The Ps2 Gene And In Its Expression,” in Human Breast Cancer Cell Lines, Gene, 1995 May 19; 157 (1-2): 261-4; WO 97/46705 and WO 95/15373).

A frequently used method for the analysis of the methylation status of specific CpG positions is based on the described specific reaction of bisulfite with cytosine is the so-called methylation-sensitive PCR (MSP) (Herman, J. G., Graff, J. R., Myohanen, S., Nelkin, B. D., Baylin, S. B., 1996, “Methylation-Specific PCR: A Novel PCR Assay for Methylation Status of CpG Islands,” Proc. Natl. Acad. Sci., USA, Sep. 3; 93 (18): 9821-6). However, if amplificate is not obtained with the use of primers designed to specifically hybridize to initially methylated cytosines, MSP cannot determine whether the DNA sample was originally unmethylated or the amplification failed to work. An internal control that can differentiate between these two alternatives is not known in the prior art.

MSP utilizes primers that hybridize to a sequence formed by the bisulfite treatment of a DNA sample that is either not methylated at the respective position, or to a nucleic acid formed by the bisulfite treatment of a DNA that is methylated at the respected position. With these primers, amplified products can then be produced, whose detection in turn suggests the presence of a methylated or unmethylated position in the sample to which the primers bind.

MSP is comprised of several steps. First, a bisulfite treatment is conducted, in which all cytosine bases are converted to uracil bases while the methylated cytosine bases (5-methylcytosine) remain unchanged. In the next step, primers that are complementary to a methylated DNA converted with bisulfite but not complementary to a corresponding DNA that was originally in the unmethylated state are used to selectively amplify the originally methylated DNA. However, if amplificate is not obtained, the prior art does not include an internal control that distinguishes whether the DNA sample was originally unmethylated or the amplification failed to work. With regard to the primer design on bisulfite treated nucleic acids the state of the art is summarized in the following references:
Li LC, Dahiya R. (2002) “MethPrimer: designing primers for methylation PCRs.” Bioinformatics 2002 Nov; 18(11):1427-31.) and
<www3.mdanderson.org/leukemia/methylation/msp.h tml>, “Methylation Specific PCR,” Herman, James, in “CpG Island Methylation in Aging and Cancer,” with reference to Herman, J G, and Baylin, S B, “Methylation Specific PCR,” in Current Protocols in Human Gemetics (1998).

PCR, be it methylation specific PCR as described by Herman or conventional PCR, has several disadvantages. Quantification requires either multiple samples or aliquots taken from a single sample at intervals. The amplification product then has to be detected by techniques such as gel electrophoresis, ethidium bromide staining, or Southern blotting. However, preparation of multiple samples is expensive, and withdrawal of aliquots from a single sample often leads to contamination. Moreover, agarose gel analysis lacks sensitivity and specificity, and Southern blotting is laborious. The accuracy of any of these techniques is limited.

With Real Time PCR however, these drawbacks to quantification are at least partially eliminated. Real-time quantitative PCR is a homogeneous method that includes both amplification and analysis with no need for slab gels, radioactivity or sample manipulation. There are now several platforms commercially available for combining thermal cycling with fluorescence acquisition (Foy C A, Parkes H C. Emerging homogeneous DNA-based technologies in the clinical laboratory. Clin. Chem. 2001; 47: 990-1000) . The LightCycler™ instrument from Roche, for example, is specially designed for online quantification in real-time. It comprises a thermal cycler, combined with a microvolume fluorimeter.

More recent methods for the detection of cytosine methylation are also based on quantitative PCR (“Real-Time Quantitative PCR,” Heid et al., Genome Res. 6:986-994, 1996; Gibson et al., Genome Res. 6:995-1001, 1996) (e.g., “TaqMan™,” “LightCycler™,” and “Sunrise™” technologies).

The methylation specific application analogons of the TaqMan™ and LightCycler™ technologies, have become known as “MethyLight” (WO 00/70090), the sequence discrimination can occur at either or both of two steps: (1) the amplification step, or (2) the fluorescence detection step.

In the MethyLight technology, sequence discrimination at the PCR amplification level occurs by designing the primers and probe or just primers to overlap potential sites of DNA methylation (CpG dinucleotides). Each oligonucleotide can cover anywhere from zero to multiple CpG dinucleotides. Each CpG dinucleotide can result in two different sequence variations following bisulfite conversion, depending on whether that particular site was methylated or unmethylated. For example, if an oligonucleotide overlaps two CpG dinucleotides, then the number of possible sequence variants in the genomic DNA within this region covered by that oligonucleotide is 22=4. If both of the primers and the probe each overlap two CpGs, then the total number of variants contained within the sequence covered by the oligonucleotides is 4×4×4=64. In theory, one could design separate PCR reactions to analyze the relative amounts of each of these potential 64 sequence variants.

However, significant methylation information can be derived from the analysis of a much smaller number of variants by designing reactions for the fully methylated and fully unmethylated molecules, which represent the two most extreme sequence variants. The ratio between these two reactions or, more reliably, the ratio between the methylated reaction and a control reaction would provide a measure for the prevalence of methylated molecules at this locus.

The MethyLight technology can also be modified to avoid sequence discrimination at the PCR amplification level. If neither the primers nor the probe overlie any CpG dinucleotides, then the reaction represents unbiased amplification and can serve as a control for the amount of input DNA. The ideal control reaction is one in which the entire amplicon is devoid of any CpG dinucleotides in the unconverted genomic sequence.

When just the probe is designed to cover CpG dinucleotides, then sequence discrimination occurs solely at the level of probe hybridization. In this version, all sequence variants resulting from the sodium bisulfite conversion step are amplified with equal efficiency, as long as there is no amplification bias. In this case, the design of separate probes for each of the different sequence variants associated with a particular methylation pattern (22=4 probes in the case of two CpGs) would allow a quantitative determination of the relative prevalence of each sequence permutation in the mixed pool of PCR products.

Different types of probes or detection systems can be employed to analyse methylation at specific CpG positions with the use of MethyLight. One possibility is the Taqman system. TaqMan™ is a homogenous amplicon detection system that uses TaqMan™ polymerase. This enzyme does not possess a 3′-5′ exonuclease activity, but is 5′-3′ exonucleolytic. These properties form the basis of a 5′ exonuclease assay that detects target DNA as the PCR proceeds in real time. TaqMan™ functions by including an oligonucleotide probe designed to hybridize to a GC-rich sequence located between the forward and reverse primers.

TaqMan™ probes are blocked from extension at their 3′ terminus and are labeled with a fluorescent reporter at the 5′ terminus. The probes are also conjugated to another fluorophore, which quenches the fluorescence of the reporter when both labels are in close proximity. Degradation of the probes from their 5′-end liberates label; therefore, TaqMan™ specificity results from the probes annealing to their amplicon, followed by their cleavage to separate the reporter and quencher fluorophores. This separation gives rise to an increase in fluorescence when appropriately illuminated (Whitcombe et al., “A Homogeneous Fluorescence Assay for PCR Amplicons: Its Application to Real-Time, Single-Tube Genotyping,” Clinical Chemistry. 1998; 44: 918-923).

In the case of the “Sunrise™” technology, the amplification and fluorescent steps are the same and the fluorescence is generated by the primer itself. In the case of the FRET hybridization, either or both of the FRET oligonucleotides can be used to distinguish the sequence difference. It is possible with this method to detect the methylation status of individual positions or a few positions directly in the course of the PCR, so that a subsequent analysis of the products is spared.

Another suitable probe system would be the LightCycler system. A LightCycler probe is a pair of single-stranded fluorescent-labeled oligonucleotides. The first oligonucleotide probe is labeled at its 3′end with a donor fluorophore dye and the second is labeled at its 5′end with an acceptor fluorophore dyes. The free 3′ hydroxyl group of the second probe is blocked with a phosphate group to prevent polymerase mediated extension.

During the annealing step of real-time quantitative PCR, the PCR primers and the LightCycler probes hybridize to their specific target regions causing the donor dye to come into close proximity to the acceptor dye. When the donor dye is excited by light, energy is transferred by Fluorescence Resonance Energy Transfer (FRET) from the donor to the acceptor dye. The energy transfer causes the acceptor dye to emit fluorescence wherein the increase of measured fluorescence signal is directly proportional to the amount of target DNA.

Returning to MSP, this method is based on the use of primer pairs wherein each primer is designed to bind to a sequence that was methylated prior to bisulfite treatment (wherein methylated cytosines remained cytosines) or unmethylated (wherein cytosines were converted to uracil) prior to bisulfite treatment. However, if there is no amplificate obtained, the result is ambiguous—i.e., the DNA sample may have been unmethylated in the original sample or the amplification may simply have failed to work. In the majority of applications this technology is used to detect methylated nucleic acids in a majority of unmethylated nucleic acids.

If both primer pairs specific for unmethylated or methylated cytosines, respectively, are used—for example, separated in different vessels or tubes for two isolated experiments or labeled with different dyes—a lack of amplification of one of the products would be accompanied by the amplification of the other product specific for the respective methylation status.

If different vessels are used for identical experiments except that each one is specific for a methylation state, theoretically an amplification product generated in only one vessel would indicate 100% methylation (or 100% non-methylation). However, in most cases a ratio of methylated versus unmethylated molecules would have to be analyzed, or the few differentially methylated molecules versus the majority of background DNA would have to be identified. Where mixtures are analyzed, both species of amplification products would be expected, therefore you expect a product in each vessel. If an amplification product is generated in only one vessel, the result would still be inconclusive and suspect, since it would not be determined whether the PCR simply failed in the other vessel.

In another scenario, two reactions could be undertaken in one vessel or tube with two different primer pairs, one primer pair specific for binding to the methylated state and another primer pair specific for binding to the unmethylated state. In this case, the lack of one amplification product in the presence of the other could be interpreted as the absence of the respective methylation state in the template and not a failure of the PCR reaction. However, if the template consists of a mix of both methylation states—which is the case for most diagnostic applications—the simultaneous amplification of both templates in one tube will result in a mix of products, which need to be differentiated from one another.

In the prior art, this problem is addressed by labeling the primers differently. If primers for the unmethylated state are labeled with one dye, for example cy-3, and primers for the methylated state are labeled with another dye, for example cy-5, the respective amplification products can be detected simultaneously and their ratio estimated. However, this approach is limited as well. The two primer pairs will likely have different melting temperatures, which affects uniform primer hybridization and will therefore most likely cause a bias towards amplification of one of the two products. It is not trivial to design methylation specific primer pairs for a duplex PCR without introducing a bias in the amplification process. Moreover, the risk of generating false PCR products from primer pairs increases, which may compete for access to the polymerase and may interfere with hybridization-based detection and failure to detect the desired amplicons. Also, bisulfite treated DNA lacks complexity, and a working MSP primer must bind to at least 2 CpG sites.

Employing two different dyes creates a further problem, which might also cause a bias towards one amplification product over the other: Different hybridizing behaviors of probes that are different only in their labels with either cy 3 or cy 5 are well known in the prior art.

### BRIEF DESCRIPTION OF THE DRAWINGS

In each of the FIGS. 1 and 2 there are 4 sections. Different primers and how they hybridize to unmethylated template is represented in section 1 and the same primers and how they hybridize to methylated template is represented in section 3. The resulting amplificates are shown in sections 2 and 4.

Legend to FIG. 1:
section 1: sequence comprising down-methylated CpGs (white ovals)
section 2: amplificate resulting from scenario in section 1
section 3: sequence comprising up-methylated CpGs (black ovals)
section 4: amplificates resulting from scenario in section 3
#2, #3=methylation unspecific primers (A-1 and B-1)
#4, #5=primers (C* and D*) comprising CG dinucleotides for detecting initially up-methylated CpGs
#19=control amplificate
#20, #21=specific amplificates indicating methylated CpGs

Legend to FIG. 2:
section 1: sequence comprising down-methylated CpGs (white ovals)
section 2: amplificate resulting from scenario in section 1
section 3: sequence comprising up-methylated CpGs (black ovals)
section 4: amplificates resulting from scenario in section 3
#8, #9, #10 and #11=methylation unspecific primers A-2, B-2, C″ and D″
#14, #15=blocking oligonucleotides (E′ and F′) comprising TG dinucleotides specific for detection of the initially un-methylated CpGs
#22=control amplificate
#23, #24=specific amplificates indicating methylated CpGs

In FIG. 1, the primers C* (# 4) and D* (# 5) are up-methylation specific. In FIG. 2, the primers C″ (# 10) and D″ (# 11) are methylation-unspecific, but blocking oligos are introduced that are specific for un-methylated template. Both cases lead to the amplification of two products in case of methylated template, and to amplification of one large fragment in case of unmethylated templates.

FIG. 1 is an illustration of an example according to one embodiment of the invention wherein said method is used to specifically detect a sequence comprising a number of methylated CpG sites (black ovals) without the ambiguity of how to interpret a lack of amplification product. In section 1 of FIG. 1 it is shown how methylation unspecific primers A (# 2) and B (#3) bind to the sequence and how the methylation specific primers C* (# 4) and D* (# 5) cannot bind to the sequence of interest comprising unmethylated positions (white ovals). In section 2 the result, one large amplificate (# 19) produced by oligonucleotide primers A and B that do not distinguish between methylated and unmethylated DNA is shown. In section 3 of FIG. 1, it is shown how primers A (# 2) and

B (# 3) bind to the sequence and how the methylation specific primers C* (# 4) and D* (# 5) (specific for methylated CpG positions (black ovals)) also bind to the sequence of interest. The result, two smaller amplificates (#20 and #21), is shown in section 4 of FIG. 1.

FIG. 2 is an illustration of an example according to one embodiment of the invention wherein said method is used to specifically detect without the use of MSP-primers a sequence comprising a number of methylated CpG sites (black ovals) without the ambiguity of how to interpret a lack of amplification product. In section 1 of FIG. 2 it is shown how primers A (# 8) and B (# 9) bind to the sequence and how the non-methylation specific primers D″ (# 11) and C″ (# 10) cannot bind to the flanking sequence downstream and upstream, respectively, of the sequence of interest comprising unmethylated CpG positions (white ovals). Their binding is hindered by the blocking oligonucleotides E′ (# 14) and F′ (#15) that hybridize to said region of interest instead, because they are designed to specifically bind to unmethylated

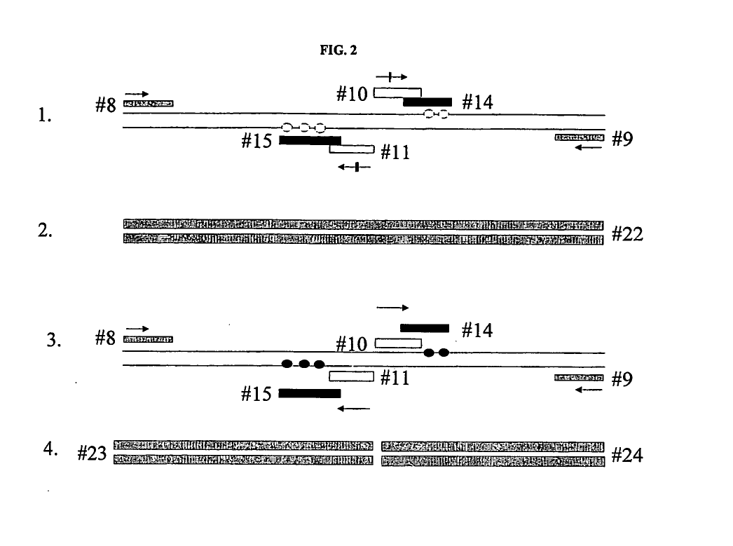

CpG positions. In section 2 of FIG. 2 the result is shown as one larger fragment (# 22) amplified by primers A (# 8) and B (# 9). In section 3 of FIG. 2 is described how the same arrangement of primers and blocking oligos binds or does not bind to an up-methylated sequence of interest. Primers A (# 8) and B (# 9) bind to the sequence and the non-methylation specific primers D″ (# 11) and C″ (# 10) also bind to the flanking sequence downstream and upstream, respectively, of the sequence of interest comprising said methylated CpG positions (black ovals), resulting in the amplification of two smaller fragments (#23 and # 24).

### DESCRIPTION OF ILLUSTRATIVE EMBODIMENTS

The current invention discloses a method for methylation-specific amplification of a sequence in which the method does not depend on the design of two primer pairs one wherein both primers bind specifically to methylated and one wherein both primers bind specifically to unmethylated CpG sites, or on the use of different dyes in one vessel with the risk of introducing a bias to the amplificates. The invention overcomes the ambiguity of the lack of an amplification product due to the lack of the template or due to the failure of the PCR. Most important, the invention introduces a mean to control the performance of a PCR reaction simultaneously within a single tube at the exact same region of nucleic acid which is analyzed by the PCR tested. That way, it is less likely that the amplification of a control fragment (control PCR) behaves differently than the amplification of interest. The risk of introducing a bias is limited to a minimum. It is very advantageous that the control takes place in the same tube as the original reaction, as the risk of contamination (for example carry over contamination) is minimized.

Furthermore the invention is easily applicable. It does not require great efforts to design appropriate primers and no additional costs for labels (fluorescent label) occur, if the result does not need to be quantitative, which makes the basic method an attractive alternative to RealTime PCR methods.

According to an embodiment of the current invention, several fragments are produced in the amplification step in one tube, one such fragment being produced with methylation unspecific primers and one or two additional fragments being produced with methylation specific primers, as outlined in the drawings. A ‘non-methylation specific primer’ (non-MSP primer) is understood to be a primer that does not comprise a CG (or TG when it was a unmethylated CG prior to bisulfite conversion) dinucleotide sequence. If the methylation specific primers bind to the template, two products will be formed as the two MSP primers are designed to bind “back-to-back” onto the template DNA and build a primer pair with a non-MSP primer upstream and downstream respectively. Template molecules with a methylation status opposite to the one the primers were designed for will still serve as template for the non-MSP primers and allow for the amplification of a larger control fragment. An amplificate is produced in any case on the very same genomic fragment, serving as a positive control. The results can be easily interpreted from a single gel lane, as the fragment sizes and number of bands are different depending on methylation status.

If amplificates are produced, then the methylation status is confirmed. If the expected methylation state is detected, two smaller amplification products are formed, one of which confirms the other; if not, only one larger fragment is formed. Heterogeneous methylation can be identified if only one of the smaller fragments is formed. The results can be easily interpreted from a single gel lane, as the fragment sizes and number of lanes are different depending on methylation status. Only two primer pairs are needed for the independent analysis of two methylatable positions, and no additional primers are needed for the control fragment. If amplification products are produced, it is confirmed that the PCR worked, and an unambiguous conclusion about the methylation status is possible.

Additionally, if no amplificates are produced it is confirmed that the PCR failed.

Unlike duplex MSP-PCR, the first set of primers (of the kind A and B), which serve as a pair if the larger control fragment is produced, are methylation unspecific and therefore much easier to design. Also, bias is not an issue, since no ratio has to be determined between products amplified in one case and in the other case.

In an embodiment of the invention, the method can also be used to analyze cases where the template is not co-methylated. In such a case it is possible that only one of the two MSP primers binds to the template DNA and leads to the amplification of only one smaller fragment.

The method may also be used for quantification of a ratio of methylated versus non-methylated DNA, in which case the ratio between the large fragment and the two smaller fragments can be determined, although the amplification may be biased towards the amplification of two smaller fragments versus one large fragment.

The object of the present invention is to overcome a disadvantage of the prior art and to provide an improved method for the detection of cytosine methylation including an internal control. This aim is to be achieved with methylation specific primers that lead to the amplification of two smaller fragments instead of one big fragment whenever a specific state of methylation is present, and further lead to a different product, larger in size, if this methylation state is not given. Another advantage of this invention is that an amplification product is formed in any case, thus providing a control that the amplification reaction in question works at all. It is especially advantageous that said control fragment is located at the very same region of the nucleic acids to be analyzed. Other advantages may become apparent to those skilled in the art, and the above recitation of advantages is not meant to be restrictive.

As used herein, “nucleic acid” refers to deoxyribonucleotides or ribonucleotides and polymers thereof in either single- or double-stranded form. The term encompasses nucleic acids containing known nucleotide analogs or modified backbone residues or linkages, whether synthetic, naturally occurring, or non-naturally occurring, and which have similar binding properties as the reference nucleic acid and are metabolized in a manner similar to the reference nucleotides. Examples of such analogs include, without limitation, phosphorothioates, phosphoramidates, methyl phosphonates, chiral-methyl phosphonates, 2-O-methyl ribonucleotides.

A “converted nucleic acid” is a chemically treated nucleic acid, for example with sodium bisulfite, followed by alkaline treatment so that unmethylated cytosines are converted to uracil, while methylated cytosines are left intact.

A “treated nucleic acid sample” refers to a nucleic acid sample that has been treated with an agent which converts unmethylated cytosine bases within said nucleic acid sample into uracil bases and which does not change methylated cytosines within said nucleic acid sample. The terms “initially methylated” or “initially unmethylated” refer to the methylation states of nucleic acids prior to treatment with an agent which converts unmethylated cytosine bases in into uracil bases and which does not change methylated cytosines within said nucleic acid sample.

A “methylation specific primer” refers to a primer oligonucleotide for use in the methylation discriminating amplification of a bisulfite treated nucleic acid, wherein the binding site of the primer on the nucleic acid contains at least one CpG position.

“Secondary structures” of oligonucleotide primers:
A “hairpin structure” or a “stem” refers to a double-helical region formed by base pairing between adjacent, inverted, complementary sequences in a single strand of DNA.
A “stem-loop” structure refers to a hairpin structure, further comprising a loop of unpaired bases at one end.

The term “molecular beacon” refers to a molecule capable of participating in a specific binding reaction and whose fluorescent activity changes when the molecule participates in that binding reaction.

Embodiments of the invention are directed to a method, to a kit, and to primers for the detection of cytosine methylation in a nucleic acid sample in which unmethylated cytosine bases in the nucleic acid sample are converted into uracil bases using a converting agent that does not change methylated cytosines in said nucleic acid sample. Selected segments of the converted nucleic acid sample are amplified utilizing two oligonucleotide primers, A and B, that are capable of forming an amplificate under chosen amplification conditions and do not distinguish between initially methylated and unmethylated DNA. Further, at least two additional second oligonucleotide primers C and D are provided that can form products under the same amplification conditions chosen above with one of the primers A or B. Preferably these second primers (C and D) bind to DNA in a methylation specific manner. Said preferred second primers (C and D) thereby distinguish between initially methylated and unmethylated DNA.

Alternatively, methylation sensitive blocking molecules in the same reaction mixture hinder the binding of one or both of the second primers (C or primer D), which in that case are preferably non-methylation specific, to nucleic acids in a methylation specific manner, thereby distinguishing between initially methylated and unmethylated nucleic acids. The amplificates of the converted nucleic acids are detected, allowing conclusions to be drawn concerning the degree of DNA methylation at different CpG positions and the presence of a disease or another medical condition of the patient.

The blocking oligonucleotides are modified at the 3′ end by a phosphate group in order to prevent their elongation during the PCR. The amplification is performed in the presence of CpG specific blocking oligonucleotides, capable of distinguishing between unmethylated and methylated nucleic acid. The amplification of a larger fragment enabled by primers A and B is not hindered by the blocking oligos binding to CpG positions in the region of interest, because the blocking oligos are degraded. This means they are removed by the polymerase exonuclease activity, if the amplifying enzyme has already started copying the template enabled by the successful binding of one of the primers A or B. Only when the binding of primers of the kind C and D is hindered by said blocking oligos, the amplifying enzyme cannot start the copying process of the smaller fragments. But when binding between the primet pairs of a given fragment, like A and B, degradation of said blocking oligos takes place and amplification is not signi [symbol] as it is directed to a method can be described as a method for the detection of cytosine methylation in a nucleic acid sample, comprising the steps of
a) treating a nucleic acid sample with an agent converting unmethylated cytosine bases into uracil bases and not converting methylated cytosine bases within said nucleic acid sample,
b) amplifying selected segments of the treated nucleic acid sample, by providing two first oligonucleotide primers (A and B) that are capable of producing an amplificate under certain chosen amplification conditions independently of the methylation status of the nucleic acid before treatment in step a), and further providing at least two additional second oligonucleotide primers (C and D) that can each produce a product with one of the first primers (A or B) under said same amplification conditions,
   wherein at least one of the second primers binds to the nucleic acid in a methylation specific manner, thereby distinguishing between unconverted initially methylated and converted unmethylated nucleic acids
   and/or blocking molecules are provided that hinder the binding of at least one of the second primers to the nucleic acid in a methylation specific manner, thereby distinguishing between unconverted initially methylated and converted unmethylated nucleic acids,
c) detecting the amplificates of the treated nucleic acid.

It is a preferred embodiment of teh invention that an additional step is performed,
d) analysing the size of the detected amplificates.
   It is also preferred that said selected fragments of the treated nucleic acids are amplified using PCR. It is a preferred embodiment that the second set of primers (C and D) is methylation specific. It is also a preferred embodiment that these primers show ho mismatches. It is preferred that the hybridisation takes place as a 100% matching hybridisation. Three different scenarios are preferred, firstly, either both (or all) primers of the second set are specific for initially methylated nucleic acids, that means each of the second primer binds to a sequence containing at least one CpG position that was methylated prior to treatment in step (a) or, secondly, the each of the second set of primers (C and D) binds to a sequence containing at least one CpG position that was unmethylated prior to treatment in step (a), or, thirdly, one of the primers from the second primer set (for example C) binds to a sequence containing at least one CpG position that was methylated prior to treatment in step (a) whereas other primers of that second set (for example primer D) bind to a sequence containing at least one CpG position that was unmethylated prior to treatment in step (a).

It is furthermore preferred that one of the blocking molecules hinders the binding of one primer of the second set of primers (for example C) and the other one hinders the binding of another primer of the second set of primers (for example D).

It is especially preferred that a blocking molecule binds to a sequence comprising at least one CpG position that was methylated prior to treatment in (a) and thereby hinders the amplification of a segment by means of the second primer whenever said CpG is methylated before treatment in (a).

In an embodiment according to the invention, a nucleic acid sample, illustrated here by DNA, is obtained from cell lines, tissue embedded in paraffin, for example, tissue from eyes, intestine, kidneys, brain, heart, prostate, lungs, breast or liver, histological slides, or bodily fluids or any combinations thereof.

The term ‘bodily fluid’ is meant to describe one or several of the following sources of nucleic acids: whole blood, blood plasma, blood serum, urine, stool, sputum, ejaculate, semen, tears, sweat, saliva, lymph fluid, bronchial lavage, pleural effusion, peritoneal fluid, meningal fluid, amniotic fluid, glandular fluid, fine needle aspirates, nipple aspirate fluid, spinal fluid, conjunctival fluid, vaginal fluid, duodenal juice, pancreatic juice, bile, amniotic fluid and cerebrospinal fluid.

Genomic DNA can be obtained from DNA of cells, tissue or other test samples by standard methods, as found in references such as Sambrook, Fritsch and Maniatis, Molecular Cloning: A Laboratory Manual, CSH Press, 2nd edition, 1989: Isolation of genomic DNA from mammalian cells, Protocol I, p. 9.16-9.19 and in the commonly used QIAamp DNA mini kit protocol by Qiagen. Conversion of unmethylated, but not methylated, cytosine bases within the DNA sample is conducted with a converting agent, preferably a bisulfite such as disulfite or hydrogen sulfite. In a preferred embodiment, the conversion is conducted after embedding the DNA in agarose. A reagent that denatures the DNA duplex is also present for the conversion, and may be combined with a radical scavenger.

Selected segments of the converted nucleic acid sample are amplified preferably by polymerase chain reaction technique (PCR). Two oligonucleotide primers A and B are used for forming an amplificate, which do not distinguish between initially methylated and unmethylated DNA.

Within this description the methylation unspecific primer nucleotides, which enable the amplification of one large control fragment, are generally named A and B or first oligonucleotide primers. (In the illustrated examples when these primers refer to specific sequences, which differ in the two examples these primers are referred to as A-1 and B-1 or A-2 and B-2 respectively, and are introduced simply to avoid confusion upon the sequence differences.)

When a primer pair is named C and D, be it C*, C′ or C″ or be it D*, D′ or D″ it is referred to its general position, that is “back-to-back” to each other and located ‘inside’ of the two primers A and B.

C* and D* refer to said primer molecules when they are designed to hybridize to an initially methylated template, they comprise CG dinucleotides. C′ and D′ refer to said primer molecules whenever they are designed to hybridize to an initially unmethylated template, they comprise at least one TG or CA dinucleotide. C″ and D″ refer to said primer molecules when they are designed to not differentiate between initially unmethylated and methylated template nucleic acid.

The same nomenclature is used to differentiate between blocking oligo nucleotides E and F that hybridize methylation specific to the template nucleic acids. E* and F* blocking oligos are specifically hybridizing to initially methylated template nucleic acids and E′ and F′ blocking oligos are specifically hybridizing to initially unmethylated template nucleic acids.

At least two additional oligonucleotide primers C and D capable of forming products with one of the primers A or B are also utilized. Both primers C* and D* (or C′ and D′) bind to DNA in a methylation specific manner and thereby distinguish between initially methylated and unmethylated DNA. Alternatively, blocking molecules hinder the binding of primers C″ and D″ or they hinder the binding of one primer, either C″ or D″, to DNA in a methylation specific manner, thereby distinguishing between initially methylated and unmethylated DNA.

In a preferred embodiment, one primer, for example primer C*, binds to at least one CpG position in a nucleic acid sample that was initially methylated (i.e., methylated before bisulfite treatment), and the other primer, primer D′ binds to at least one CpG position in a nucleic acid that was initially unmethylated in the sequence the primer is hybridizing to.

In another embodiment, primer C* and primer D* each binds to at least one CpG position in a nucleic acid that was initially methylated in the sequence the primer is hybridizing to.

In yet another embodiment, primer C′ and primer D′ each binds to at least one CpG position in a nucleic acid that was initially unmethylated in the sequence the primer is hybridizing to.

In another embodiment, one or both of primers C* and D* (or C′ and D′) bind(s) to a sequence containing initially methylated (or unmethylated) CpG dinucleotides in the sequence the primer is hybridizing to.

In addition, in another embodiment, a blocking molecule hinders the binding of primer C″, while another blocking molecule hinders the binding of primer D″. In another embodiment, the blocking molecules hinder the binding of one or both of primers C* and D* or C′ and D′ to at least one CpG position in a nucleic acid of the sequence that was initially methylated or unmethylated.

In a preferred embodiment, methylation specific primers C and D result in the amplification of two small fragments whenever a specific state of methylation is present, thereby introducing a control for methylation-sensitive PCR. In the absence of said methylation state the oligonucleotides C and D cannot serve as primers due to the fact that they are not binding to the template, the primers A and B, however, which do not distinguish between initially methylated and unmethylated DNA, result in amplification of one large fragment, thereby introducing a negative control for methylation-specific PCR. By comparing the amount of amplificates formed, it is possible to normalize the level of methylation at a particular position to the level of non-methylation.

In a preferred embodiment of the present invention, amplification and detection occur simultaneously as measured by fluorescence-based real-time quantitative PCR (Heid et al., Genome Res., 6:986-994, 1996) using one or a plurality of specific oligonucleotide probes, where at least one oligonucleotide probe is a CpG specific probe capable of distinguishing between unmethylated and methylated nucleic acid.

It is therefore particularly preferred that the amplification and detection steps comprise fluorescence-based quantitative PCR and it is furthermore preferred that the amplification is performed in the presence of one or a plurality of specific oligonucleotide probes, wherein at least one of said oligonucleotide probes is a CpG specific probe capable of distinguishing between unmethylated and methylated nucleic acids.

It is particularly preferred that a LightCycler assay is conducted, in which a fluorescence change occurs during the PCR. Additional fluorescent-labeled oligonucleotides can be added to the oligonucleotides in a preferred amplification of the DNA, and the change in fluorescence is measured during the PCR reaction. Since the DNA is amplified, information on the methylation status of different CpG positions can be obtained directly from this change in fluorescence. Since different oligonucleotides are each preferably provided with different fluorescent dyes, a distinction of the change in fluorescence during the PCR is also possible, separately for different positions. The fact is utilized that a fluorescence resonance energy transfer (FRET) can occur between two dyes. only if they are found in the direct vicinity of one another, i.e., about 1-5 nucleotides apart. Then the second dye can be excited by the emission of the first dye and in turn emit light of another wavelength, which is then detected.

In a preferred embodiment,' the probe further comprises one or a plurality of fluorescent-labeled moieties. It is also preferred that a TaqMan™ assay is conducted, the methylation specific variation of which has become known as “methyl-light” (WO 00/70090). It is possible with this method to detect the methylation status of individual positions or a few positions directly in the course of the PCR, so that a subsequent analysis of the products is not required.

It is also preferred that fluorescent molecular beacons are used for the detection (WO 00/46398).

It is particularly preferred that the amplification process employed in all inventive embodiments herein may be fluorescence based Real Time Quantitative PCR (Heid et al., Genome Res., 6:986-994, 1996) employing a dual-labeled fluorescent oligonucleotide probe (TaqMan™ PCR, using an ABI Prism 7700 Sequence Detection System, Perkin Elmer Applied Biosystems, Foster City, Calif.). The detection method may comprise measuring a fluorescence signal based on the amplification-mediated displacement of the CpG-specific probe.

It is also particularly preferred that a Sunrise™/amplifluor probe (U.S. Pat. No. 6,350,580) is employed.

It is also preferred that the degree of methylation in at least one selected segment of the nucleic acids is determined based on the ratio of the different abundance of the amplificates obtained. It is also preferred that the methylation status of the nucleic acid sample is deducted from the size of the detected amplificates.

In a preferred embodiment of the invention, the additional second oligonucleotide primers (C or D) have a secondary structure, as defined herein, including a stem loop, a hairpin, an internal loop, a bulge loop, a branched structure, and a pseudoknot, or multiple secondary structures—cloverleaf type structures or any three-dimensional structure. This secondary structure changes upon binding of the oligonucleotide primer pairs to the treated nucleic acids.

Preferably, the analysis (described as a preferred additional step of the method) is conducted by measurement of the length of the amplified nucleic acids, especially DNA, whereby methods of length measurement include gel electrophoresis, capillary gel electrophoresis, chromatography (e.g. HPLC), and other suitable methods known to those skilled in the art.

In a preferred embodiment, the presence of a disease or another medical condition of the patient is concluded from the degree of methylation at different CpG positions investigated.

In another embodiment, a method according to the invention is used for distinguishing cell types or tissues or for investigative cell differentiation. In yet another embodiment, a method according to the invention is used for the detection of a disease within a subject or tissue.

A preferred embodiment is a kit comprising primers A, B, C and D, wherein the primers C and D are either C* and D* or C′ and D′ primers, and wherein primers A and B are capable to form an amplificate from a converted nucleic acid sample serving as the template under chosen amplification conditions, and do not distinguish between initially methylated and unmethylated DNA. Said primers C and D of the kit can each form a product with the primers B and A respectively from a converted DNA sample serving as the template under the chosen amplification conditions, wherein primers C* and D* or C′ and D′ bind to DNA in a methylation specific manner, thereby distinguishing between initially methylated and unmethylated DNA.

Generally this preferred embodiment is a kit comprising primers of four types, A, B, C and D, wherein primers A and B are capable of producing an amplificate from a bisulfite treated template nucleic acid sample, under suitable amplification conditions, and do not distinguish between prior to bisulfite treatment methylated and prior to bisulfite treatment unmethylated DNA, and wherein primers C and D can produce amplificates with one of the primers A or B from a bisulfite treated DNA sample serving as the template under said amplification conditions and wherein at least one of the primers primers C and D binds to DNA in a methylation specific manner, thereby distinguishing between prior to bisulfite treatment methylated and prior to bisulfite treatment unmethylated DNA.

Further to a preferred kit comprising primers of the kind A, B, C and D, the kit may also comprise of one or two blocking oligonucleotides of the kind E and F. Primers A and B are capable to form an amplificate from a converted nucleic acid sample serving as the template under certain chosen amplification conditions, and do not distinguish between initially methylated and unmethylated DNA. Primers C and D can each form a product with the primers B and A respectively from a converted DNA sample serving as the template under said certain amplification conditions chosen, preferably without specificity towards one methylation state. Blocking nucleotides E and F bind to DNA in a methylation specific manner (either they are methylation specific E* and F* or un-methylation specific E′ and F′) and hinder the binding of at least one of the primers C or D to the modified DNA sample, thereby distinguishing between initially methylated and unmethylated DNA.

Generally a kit is preferred comprising primers A, B, C and D, wherein primers A and B are capable of producing an amplificate from a bisulfite treated template nucleic acid sample, under suitable amplification conditions, and do not distinguish between prior to bisulfite treatment methylated and prior to bisulfite treatment unmethylated DNA, and wherein primers C and D can produce amplificates with one of the primers A or B from a bisulfite treated template DNA sample under said amplification conditions, and further comprising blocker molecules that bind to the bisulfite treated nucleic acid in a methylation specific manner and thereby hinder the binding of at least one of the primers C or D to the DNA sample, thereby distinguishing between prior to bisulfite treatment methylated and prior to bisulfite treatment unmethylated DNA.

Preferred is also a kit, characterized in that it additionally contains instructions for conducting an assay and containers for primers of the kind of A, B, C, and D and containers for a polymerase and nucleotides to conduct a PCR reaction with these primers according to the instructions.

### EXAMPLES

The following examples explain the invention:

In the following examples the methylation status of CpGs in the promoter region of the upmethylated bisulfite treated sequence of the GSTP1 gene (as disclosed in SEQ. ID No. 1) was analyzed in samples of cancer patients. The resulting amplificates were detected with gel electrophoresis, in a single lane in which the amplified products separated. Alternatively a real time methylation specific PCR was carried out using bisulfite treated DNA as the template and fluorescent labeled probes covering CpG positions of interest. The latter experiment is not described in detail herein.

Upmethylated bisulfite treated sequence of the promoter region of the GSTP1 gene (SEQ. ID No. 1), 1704 by long, (accession number of the corresponding genomic sequence: NM 000852):

GGAGTTGAATAATGAGAATATATGGTTATATGGCGGCGATTAATATATAT
TGGTGTTTGTTGAGCGGGGTGTTGGGGAGGGAGAGTATTAGGAAGAATAG
TTAAGGGATATTGGGTTTAATATTTGGGTGATGGGATGATTTGTATAGTA
AATTATTATGGCGTATATATTTATGTAATAAATTTGTATATTTTGTATAT
GTATTTTAGAATTTTAAATAAAAGTTGGACGGTTAGGCGTGGTGGTTTAC
GTTTGTAATTTTAGTATTTTGGGAAGTCGAGGCGTGTAGATTATTTAAGG
TTAGGAGTTCGAGATTAGTTCGGTTAATATGGTGAAATTTCGTTTTTATT
AAAAATATAAAAATTAGTTAGATGTGGTACGTATTTATAATTTTATTTAT
TCGGGAGGTTGAAGTAGAATTGTTTGAATTCGAGAGGCGGAGGTTGTAGT
GAGTCGTCGAGATCGCGTTATTGTATTTTAGTTTGGGTTATAGCGTGAGA
TTACGTTATAAAATAAAATAAAATAATATAAAATAAAATAAAATAAAATA
AAATAAAATAAAATAATAAAATAAAATAAAATAAAATAAAATAAAATAAA
ATAAAGTAATTTTTTTTTTTTTAAGCGGTTTTTATTTTTTTTTTTTGTTT
TGTGAAGCGGGTGTGTAAGTTTCGGGATCGTAGCGGTTTTAGGGAATTTT
TTTTCGCGATGTTTCGGCGCGTTAGTTCGTTGCGTATATTTCGTTGCGGT
TTTTTTTTTGTTGTTTGTTTATTTTTTAGGTTTCGTTGGGGATTTGGGAA
AGAGGGAAAGGTTTTTTCGGTTAGTTGCGCGGCGATTTCGGGGATTTTAG
GGCGTTTTTTTGCGGTCGACGTTCGGGGTGTAGCGGTCGTCGGGGTTGGG
GTCGGCGGGAGTTCGCGGGATTTTTTAGAAGAGCGGTCGGCGTCGTGATT
TAGTATTGGGGCGGAGCGGGGCGGGATTATTTTTATAAGGTTCGGAGGTC
GCGAGGTTTTCGTTGGAGTTTCGTCGTCGTAGTTTTCGTTATTAGTGAGT
ACGCGCGGTTCGCGTTTTCGGGGATGGGGTTTAGAGTTTTTAGTATGGGG
TTAATTCGTAGTATTAGGTTCGGGTTTTCGGTAGGGTTTTTCGTTTATTT
CGAGATTCGGGACGGGGGTTTAGGGGATTTAGGACGTTTTTAGTGTCGTT
AGCGGTTTTTAGGGGGTTCGGAGCGTTTCGGGGAGGGATGGGATTTCGGG
GGCGGGGAGGGGGGGTAGGTTGCGTTTATCGCGTTTTGGTATTTTTTTTC
GGGTTTTAGTAAATTTTTTTTTGTTCGTTGTAGTGTCGTTTTATATCGTG
GTTTATTTTTTAGTTCGAGGTAGGAGTATGTGTTTGGTAGGGAAGGGAGG
TAGGGGTTGGGGTTGTAGTTTATAGTTTTTCGTTTATTCGGAGAGATTCG
AATTTTTTTATTTTTTCGTCGTGTGGTTTTTATTTCGGGTTTTTTTTTTG
TTTTTCGTTTTTTTCGTTATGTTTGTTTTTCGTTTTAGTGTTGTGTGAAA
TTTTCGGAGGAATTTGTTTATTTGTTTTTTTTTTGTATTTTTGATTTTTT
TTCGGGTTGTTGCGAGGCGGAGTCGGTTCGGTTTTTATATTTCGTATTTT
TTTTTTTTCGTAGGTCGTTGCGCGGTTTTGCGTATGTTGTTGGTAGATTA
GGGT

For the unmethylated bisulfite treated sequence of the promoter region of the GSTP1 gene, all CG dinucleotides were substituted for TG dinucleotides.

### Example 1

### Control for the Selective Amplification of GSTp1 Fragments

DNA was extracted from a sample and treated with a bisulfite solution (hydrogen sulfite, disulfite) according to the agarose bead method (such as Olek et al., Nucleic Acids Res., 1996 Dec 15;24(24):5064-6.). The treatment is such that all non-methylated cytosines within the sample are converted to thymine, whereas 5-methylated cytosines within the sample remain unmodified.

The developed assay for GSTp1 was tested with a methylated control sample using the following primers:

| Primer A-1: | |
|---|---|
| 5′-ATTTGGGAAAGAGGGAAAG-3′; | (SEQ. ID: 2) |
| found at position 792 of the GSTP1 promoter | |
| region used. | |
| Primer B-1: | |
| 5′-CCCTACCAAACACATACTCC-3′; | (SEQ. ID: 3) |
| found at position 1373 of the GSTP1 promoter | |
| region used. | |
| Primer C*: | |
| 5′-GTATTAGGTTCGGGTTTTCG-3′; | (SEQ. ID: 4) |
| found at position 1110 of the GSTP1 promoter | |
| region used. | |
| Primer D*: | |
| 5′-TAATAACGAAAACTACGACGAC-3′; | (SEQ. ID: 5) |
| found at position 1022 of the GSTP1 promoter | |
| region used. | |

The developed assay for GSTp1 was tested with an unmethylated control sample using the following primers:

| Primer A-1: | |
|---|---|
| 5′-ATTTGGGAAAGAGGGAAAG-3′; | (SEQ. ID: 2) |
| found at position 792 of the GSTP1 promoter | |
| region used. | |
| Primer B-1: | |
| 5′-CCCTACCAAACACATACTCC-3′; | (SEQ. ID: 3) |
| found at position 1373 of the GSTP1 promoter | |
| region used. | |
| Primer C′: | |
| 5′-GTATTAGGTTTGGGTTTTTG-3′; | (SEQ. ID: 6) |
| found at position 1110 of the GSTP1 promoter | |
| region used. | |
| Primer D′: | |
| 5′-TAATAACAAAAACTACAACAAC-3′; | (SEQ. ID: 7) |

found at position 1022 of the GSTP1 promoter
region used.

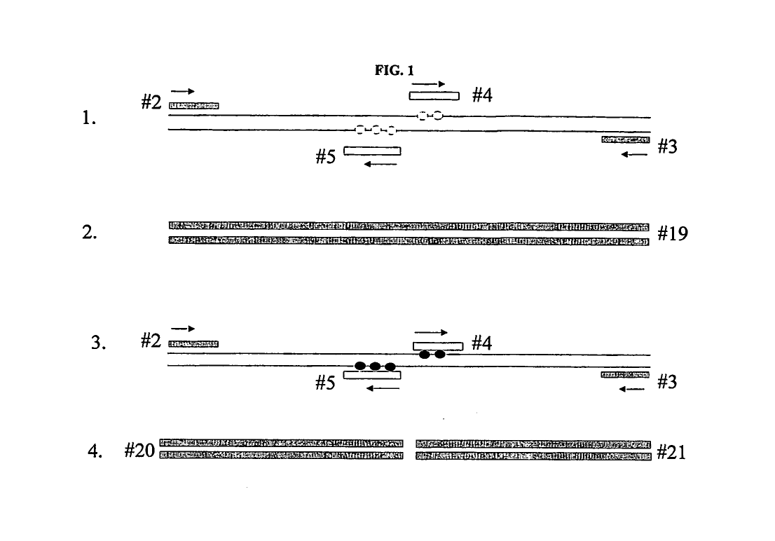

A selected fragment of the converted GSTp1 sequence was amplified with the oligonucleotide primers A-1 (SEQ. ID: 2) and B-1 (SEQ. ID: 3) that do not distinguish between initially methylated and unmethylated DNA. The additional oligonucleotide primers C* and D* can form amplification products with B-1 (SEQ. ID: 3) and A-1 (SEQ. ID: 2) respectively. Generally, primer A-1 (SEQ. ID: 2) is able to form an amplificate with primer D* (SEQ. ID: 5) and primer C* (SEQ. ID: 4) is able to form an amplificate with primer B-1 (SEQ. ID: 3) given that the primers bind to the template. The primers C* (SEQ. ID: 4) and D* (SEQ. ID: 5) that are specific for the methylated state of the GSTp1 sequence (concerning formation of a PCR product with primers A-1 (SEQ. ID: 2) and B-1 (SEQ. ID: 3) lead to the amplification of two small fragments when respective sites are methylated as shown in FIG. 1, # 4.

The oligonucleotide primers A-1 (SEQ. ID: 2) and B-1 (SEQ. ID: 3) that do not distinguish between initially methylated and unmethylated DNA of the methylated state of the GSTp1 sequence lead to the amplification of one large fragment when respective sites are unmethylated, provided that primers C* (SEQ. ID: 4) and D* (SEQ. ID: 5) are specific for upmethylated CpGs (FIG. 1, # 1 and # 2).

Therefore, if the sample DNA was initially unmethylated, a longer fragment is formed by primers A-1 (SEQ. ID: 2) and B-1 (SEQ. ID: 3), 601 bp long, whereas if the sample was initially methylated at the sites covered by primers C* (SEQ. ID: 4) and D* (SEQ. ID: 5) two smaller fragments are produced by primer pairs A-1/D* (SEQ. ID: 2 and SEQ. ID: 5), 253 bp long, and C*/B-1 (SEQ. ID: 4 and SEQ. ID: 3), 282 bp long. Depending on the degree of methylation in a given sample, all three fragments may be observed.

The PCR was carried out in a reaction volume of 25 μl in a standard thermocycler (Mastercycler, Fa. Eppendorf). Each PCR reaction mixture consisted of 500 nM of each primer (MWG, Germany); 1 unit of HotStar-Taq polymerase (Qiagen); 1× reaction buffer including 1.5 mM MgCl2 (Qiagen), 250 μM each of dATP, dCTP, dGTP and dTTP (Fermentas); 5 μl (10 ng) of bisulfite treated DNA solution were used in each reaction. Thermal cycling was initiated with a first denaturation step of 95° C. for 15 min. The thermal profile for the PCR was 95° C. for 30 s; 56° C. for 1 min and 72° C. for 1 min for 40 cycles. 10 μl of PCR products were analyzed performing a gel electrophoresis in a 1.5% agarose gel including ethidium bromid as staining agent.

The primers C′ (SEQ. ID: 6) and D′ (SEQ. ID: 7) that are specific for the unmethylated state of the GSTp1 sequence (in combination with primers A-1 (SEQ. ID: 2) and B-1 (SEQ. ID: 3) as seen above) lead to the amplification of two small fragments when respective sites are unmethylated (no figure).

The oligonucleotide primers A-1 (SEQ. ID: 2) and B-1 (SEQ. ID: 3) that do not distinguish between initially methylated and unmethylated DNA of the unmethylated state of the GSTp1 sequence lead to the amplification of one large fragment when respective sites are methylated, provided that primers C′ (SEQ. ID: 6) and D′ (SEQ. ID: 7) are specific for unmethylated CpGs.

Therefore, if the sample DNA was initially methylated, a longer fragment is formed by primers A-1 (SEQ. ID: 2) and B-1 (SEQ. ID: 3), 601 bp long, whereas if the sample was initially unmethylated at the sites covered by primers C′ (SEQ. ID: 6) and D′ (SEQ. ID: 7), two smaller fragments are produced by primer pairs A-1/D′, (SEQ. ID: 2 and SEQ. ID: 7) 253 bp long, and C′/B-1 (SEQ. ID: 6 and SEQ. ID: 3) 282 bp long. Depending on the degree of methylation in a given sample, all three fragments may be observed.

### Example 2

### Control for the Selective Amplification of GSTp1 Fragments

DNA was extracted from a sample and treated with a bisulfite solution (hydrogen sulfite, disulfite) according to the agarose bead method (Olek et al. Nucleic Acids Res. 1996 Dec 15; 24(24):5064-6.). The treatment is such that all non methylated cytosines within the sample are converted to thymidine, whereas 5-methylated cytosines within the sample remain unmodified.

With blocking oligonucleotides a higher sensitivity can be achieved, a few copies of the methylated GSTP1 promoter region can be detected in a large amount of unmethylated GSTP1 promoter region.

The developed assay for GSTp1 was tested with a methylated control sample using the following primers and blocker oligonucleotides:

(SEQ. ID: 8)

| | |
|---|---|
| Primer A-2: | 5′-GGGAAAGAGGGAAAGGTTTTTT-3′; |

found at position 796 of the GSTP1 promoter region
used.

(SEQ. ID: 9)

| | |
|---|---|
| Primer B-2: | 5′-CTAAATCCCCTAAACCCC-3′; |

found at position 1164 of the GSTP1 promoter
region used.

(SEQ. ID: 10)

| | |
|---|---|
| Primer C′′: | 5′-AGAGTTTTTAGTATGGGGTTAATT-3′; |

found at position 1082 of the GSTP1 promoter
region used.

(SEQ. ID: 11)

| | |
|---|---|
| Primer D′′: | 5′-CCCCAATACTAAATCAC-3′; |

found at position 792 of the GSTP1 promoter region
used.
Blocker oligonucleotide E* (specific for
methylated CpG):

(SEQ. ID: 12)

5′-GGTTAATTCGTAGTATTAGGTTCGGGTTTTCG-3′

Blocker oligonucleotide F* (specific for
methylated CpG):

(SEQ. ID: 13)

5′-ATACTAAATCACGACGCCGACCGCTCTTC-3′

With reference to FIG. 2, the developed assay for GSTp1 was tested with an unmethylated control sample using the following primers and blocking oligonucleotides:

(SEQ. ID: 8)

| | |
|---|---|
| Primer A-2: | 5′-GGGAAAGAGGGAAAGGTTTTTT-3′; |

found at position 796 of the GSTP1 promoter region
used.

(SEQ. ID: 9)

| | |
|---|---|
| Primer B-2: | 5′-CTAAATCCCCTAAACCCC-3′; |

found at position 1164 of the GSTP1 promoter
region used.

(SEQ. ID: 10)

| | |
|---|---|
| Primer C′′: | 5′-AGAGTTTTTAGTATGGGGTTAATT-3′; |

found at position 1082 of the GSTP1 promoter
region used.

(SEQ. ID: 11)

| | |
|---|---|
| Primer D′′: | 5′-CCCCAATACTAAATCAC-3′; |

found at position 944 of the GSTP1 promoter region
used.
Blocking oligonucleotide E′ (specific for
unmethylated CpG):

(SEQ. ID: 14)

5′-GGTTAATTTGTAGTATTAGGTTTGGGTTTTTG-3′

Blocking oligonucleotide F′ (specific for
unmethylated CpG):

(SEQ. ID: 15)

5′-ATACTAAATCACTACACCAACCACTCTTC-3′

A selected fragment of the converted GSTp1 sequence was amplified with the oligonucleotide primers A-2 (SEQ. ID: 8) and B-2 (SEQ. ID: 9) that do not distinguish between initially methylated and unmethylated DNA.

The additional oligonucleotide primers C″ and D″ (SEQ. ID: 10, SEQ. ID: 11) that do not distinguish between initially methylated and unmethylated DNA can form amplification products with B-2 (SEQ. ID: 9) and A′ (SEQ. ID: 8) respectively. Primer A-2 (SEQ. ID: 8) is able to form an amplificate with primer D″ (SEQ. ID: 11) and primer C″ (SEQ. ID: 10) is able to form an amplificate with primer B′ (SEQ. ID: 9).

The amplification of the methylated state of the GSTp1 fragment with the primer pairs C″ (SEQ. ID: 10) and B-2 (SEQ. ID: 9), and A-2 (SEQ. ID: 8) and D″ (SEQ. ID: 11) concerning formation of a PCR product is prevented by blocker oligonucleotides E or F (SEQ. ID: 12 and SEQ. ID: 13) that are specific for the methylated state in a methylation specific manner. This leads to the amplification of one large fragment.

Therefore, if the sample DNA was initially methylated, a longer fragment is formed by primers A-2 (SEQ. ID: 8) and B-2 (SEQ. ID: 9), 387 bp long, whereas if the sample was initially unmethylated at the sites covered by blocking oligonucleotides E* or F* (SEQ. ID: 12 and SEQ. ID: 13) that are specific for the methylated state two small fragments are produced by primer pair A-2/D″ (SEQ. ID: 8 and SEQ. ID: 11), 166 bp long, or C″/B-2 (SEQ. ID: 10 and SEQ. ID: 9), 100 bp long. Depending on the degree of methylation in a given sample, all three fragments may be observed.

The amplification of the unmethylated state of the GSTp1 fragment with the primer pairs C″ and B-2 (SEQ. ID: 10 and SEQ. ID: 9) and A-2 and D″ (SEQ. ID: 8 and SEQ. ID: 11) is not prevented by blocking oligonucleotides E* or F* (SEQ. ID: 12 and SEQ. ID: 13) that are specific for the methylated state. This leads to the amplification of two small fragments.

The amplification of the methylated state of the GSTp1 fragment with the primer pairs C″ and B-2 (SEQ. ID: 10 and SEQ. ID: 9) and A-2 and D″ (SEQ. ID: 8 and SEQ. ID: 11) is not prevented by blocking oligonucleotides E′ or F′ (SEQ. ID: 14 and SEQ. ID: 15), specific for the unmethylated state in a methylation specific manner. This leads to the amplification of two small fragments (FIG. 2, #4).

The amplification of the unmethylated state of the GSTp1 fragment with the primer pairs C″ and B-2 (SEQ. ID: 10 and SEQ. ID: 9) and A-2 and D″ (SEQ. ID: 8 and SEQ. ID: 11) however, is prevented by blocking oligonucleotides, specific for the unmethylated state of GSTp1, E′ (SEQ. ID: 14) or F′ (SEQ. ID: 15) in a methylation specific manner. This leads to the amplification of one large fragment (FIG. 2, #2).

Therefore, if the sample DNA was initially unmethylated, a longer fragment is formed by primers A-2 and B-2, 387 by long, whereas if the sample was initially methylated at the sites covered by blocker oligonucleotides E′ or F′ (SEQ. ID: 14 and SEQ. ID: 15) two small fragments are produced by primer pair A-2/D″ (SEQ. ID: 8 and SEQ. ID: 11), 166 bp long, or C″/B-2 (SEQ. ID: 10 and SEQ. ID: 9), 100 bp long. Depending on the degree of methylation in a given sample, all three fragments may be observed.

The blocking oligonucleotides E and F are modified at the 3′ end by a phosphate group in order to prevent their elongation during the PCR. The amplification is performed in the presence of CpG specific blocking oligonucleotides, capable of distinguishing between unmethylated and methylated nucleic acid. The amplification of a larger fragment enabled by primers A-2 and B-2 is not hindered by said blocking'oligos E and F binding to CpG positions in the region of interest, because said blocking oligos are degraded. This means they are removed by the polymerase exonuclease activity, if the amplifying enzyme has already started copying the template enabled by the successful binding of one of the primers A-2 or B-2. Only when the binding of the primers C″ and D″ is hindered by said blocking oligos, the amplifying enzyme cannot start the copying process of the smaller fragments. But when binding between the primer pairs of a given fragment, like A-2 and B-2, degradation takes place and amplification is not significantly inhibited.

1. A method for the detection of cytosine methylation in a nucleic acid sample, comprising the steps of:
a) treating a nucleic acid sample with an agent converting unmethylated cytosine bases into uracil bases and not converting methylated cytosine bases within said nucleic acid sample,
b) amplifying selected segments of the treated nucleic acid sample, by providing two first oligonucleotide primers (A and B) that are capable of producing an amplificate under certain chosen amplification conditions independently of the methylation status of the nucleic acid before treatment in step a)
and further providing at least two additional second oligonucleotide primers (C and D) that can each produce a product with one of the first primers (A or B) under said same amplification conditions,
wherein
at least one of the second primers binds to the nucleic acid in a methylation specific manner, thereby distinguishing between unconverted initially methylated and converted unmethylated nucleic acids and/or
blocking molecules are provided that hinder the binding of at least one of the second primers to the nucleic acid in a methylation specific manner, thereby distinguishing between unconverted initially methylated and converted unmethylated nucleic acids,
c) detecting the amplificates of the treated nucleic acid.

2. A method according to claim 1 further characterized in comprising the additional step
d) analysing the size of the detected amplificates.

3. A method according to claim 1, further characterized in that said selected segments of the treated nucleic acid sample are amplified using PCR.

4. A method according to claim 1, further characterized in that primers C and D each bind to a sequence containing at least one CpG position that was methylated prior to treatment in step (a).

5. A method according to claim 1, further characterized in that primers C and D each bind to a sequence containing at least one CpG position that was unmethylated prior to treatment in step (a).

6. A method according to claim 1, further characterized in that primer C binds to a sequence containing at least one CpG position that was methylated prior to treatment in step (a) whereas primer D binds to a sequence containing at least one CpG position that was unmethylated prior to treatment in step (a).

7. A method according to claim 1, further characterized in that the methylation status of the nucleic acid sample is deducted from the size of the detected amplificates.

8. A method according to claim 1, further characterized in that one of the blocking molecules hinders the binding of primer C and the other one hinders the binding of primer D.

9. A method according to claim 1 or 7, further characterized in that a blocking molecule binds to a sequence comprising at least one CpG position that was methylated prior to treatment in (a) and thereby hinders the amplification of a segment by means of the second primer whenever said CpG is methylated before treatment in (a).

10. A method according to claim 1 further characterized in that the second oligonucleotide primers have a secondary structure that changes upon binding of said second oligonucleotide primers to said treated nucleic acids.

11. A method according to claim 10, wherein said secondary structure is selected from the group consisting of a stem-loop structure, a hairpin structure, an internal loop, a bulge loop, a branched structure, a pseudoknot or a cloverleaf structure.

12. A method according to claim 1, wherein the amplification and detection steps comprise fluorescence-based quantitative PCR.

13. A method according to claim 12, wherein the amplification is performed in the presence of one or a plurality of specific oligonucleotide probes, wherein at least one of said oligonucleotide probes is a CpG specific probe capable of distinguishing between unmethylated and methylated nucleic acids.

14. A method according to claim 13, wherein the probe further comprises one or a plurality of fluorescence label moieties.

15. A method according to claim 1, 2 or 12, wherein the degree of methylation of at least one selected segment is determined based on the ratio of the different abundance of the amplificates obtained.

16. A method according to claim 1, further characterized in that the DNA samples are obtained from cell lines, whole blood, blood plasma, blood serum, urine, stool, sputum, ejaculate, semen, tears, sweat, saliva, lymph fluid, bronchial lavage, pleural effusion, peritoneal fluid, meningal fluid, amniotic fluid, glandular fluid, fine needle aspirates, nipple aspirate fluid, spinal fluid, conjunctival fluid, vaginal fluid, duodenal juice, pancreatic juice, bile, amniotic fluid and cerebrospinal fluid, and tissues, fresh frozen or embedded in paraffin, for example, tissue from eyes, intestine, colon, pancreas, kidneys, brain, heart, prostate, lungs, breast, liver or histological slides and all possible combinations thereof.

17. A method according to claim 2, further characterized in that the analysis of d) is conducted by measurement of the length of the amplified nucleic acids, whereby said method of measurement is selected from the group consisting of gel electrophoresis, capillary gel electrophoresis, chromatography (e.g. HPLC).

18. A method according to claim 1, further characterized in that the treatment performed in step a) is based on the use of a bisulfite reagent.

19. A method according to claim 18, further characterized in that the conversion is conducted after embedding the nucleic acid sample in agarose.

20. A method according to one of the preceding claims, further characterized in that the presence of a disease or other medical condition of a patient is concluded from the degree of methylation at different CpG positions investigated.

21. A kit, comprising primers A, B, C and D, wherein primers A and B are capable of producing an amplificate from a bisulfite treated template nucleic acid sample, under suitable amplification conditions, and do not distinguish between prior to bisulfite treatment methylated and prior to bisulfite treatment unmethylated DNA,
and wherein primers C and D can produce amplificates with one of the primers A or B from a bisulfite treated DNA sample serving as the template under said amplification conditions and wherein primers C and D bind to DNA in a methylation specific manner, thereby distinguishing between prior to bisulfite treatment methylated and prior to bisulfite treatment unmethylated DNA.

22. A kit, comprising primers A, B, C and D, wherein primers A and B are capable of producing an amplificate from a bisulfite treated template nucleic acid sample, under suitable amplification conditions, and do not distinguish between prior to bisulfite treatment methylated and prior to bisulfite treatment unmethylated DNA,
and wherein primers C and D can produce amplificates with one of the primers A or B from a bisulfite treated template DNA sample under said amplification conditions,
and further comprising blocker molecules that bind to the bisulfite treated nucleic acid in a methylation specific manner and thereby hinder the binding of at least one of the primers C or D to the DNA sample, thereby distinguishing between prior to bisulfite treatment methylated and prior to bisulfite treatment unmethylated DNA.

23. A kit according to one of the claim 21 or 22, characterized in that it additionally contains instructions for conducting an assay according to one of the claims 1 to 20 and containers for primers A, B, C and D and containers for a polymerase and nucleotides to conduct a PCR reaction with said primers according to said instructions.

24. The use of the method according to claim 1 for distinguishing cell types or tissues or for investigating cell differentiation.

25. The use of the method according to claim 1 for the detection of a disease within a subject or tissue.


SEQUENCE LISTING (15 sequences)

SEQ ID NO: 1 - DNA, 1704 bp, Artificial Sequence
  Upmethylated bisulfite treated sequence of the promoter region of the GSTP1 gene
ggagttgaataatgagaatatatggttatatggcggcgattaatatatattggtgtttgttgagcggggtgttggggagggagagtattaggaagaatagttaagggatattgggtttaatatttgggtgatgggatgatttgtatagtaaattattatggcgtatatatttatgtaataaatttgtatattttgtatatgtattttagaattttaaataaaagttggacggttaggcgtggtggtttacgtttgtaattttagtattttgggaagtcgaggcgtgtagattatttaaggttaggagttcgagattagttcggttaatatggtgaaatttcgtttttattaaaaatataaaaattagttagatgtggtacgtatttataattttatttattcgggaggttgaagtagaattgtttgaattcgagaggcggaggttgtagtgagtcgtcgagatcgcgttattgtattttagtttgggttatagcgtgagattacgttataaaataaaataaaataatataaaataaaataaaataaaataaaataaaataaaataataaaataaaataaaataaaataaaataaaataaaataaagtaatttttttttttttaagcggtttttattttttttttttgttttgtgaagcgggtgtgtaagtttcgggatcgtagcggttttagggaattttttttcgcgatgtttcggcgcgttagttcgttgcgtatatttcgttgcggttttttttttgttgtttgtttattttttaggtttcgttggggatttgggaaagagggaaaggttttttcggttagttgcgcggcgatttcggggattttagggcgtttttttgcggtcgacgttcggggtgtagcggtcgtcggggttggggtcggcgggagttcgcgggattttttagaagagcggtcggcgtcgtgatttagtattggggcggagcggggcgggattatttttataaggttcggaggtcgcgaggttttcgttggagtttcgtcgtcgtagttttcgttattagtgagtacgcgcggttcgcgttttcggggatggggtttagagtttttagtatggggttaattcgtagtattaggttcgggttttcggtagggtttttcgtttatttcgagattcgggacgggggtttaggggatttaggacgtttttagtgtcgttagcggtttttagggggttcggagcgtttcggggagggatgggatttcgggggcggggagggggggtaggttgcgtttatcgcgttttggtattttttttcgggttttagtaaatttttttttgttcgttgtagtgtcgttttatatcgtggtttattttttagttcgaggtaggagtatgtgtttggtagggaagggaggtaggggttggggttgtagtttatagtttttcgtttattcggagagattcgaatttttttattttttcgtcgtgtggtttttatttcgggttttttttttgtttttcgtttttttcgttatgtttgtttttcgttttagtgttgtgtgaaattttcggaggaatttgtttatttgttttttttttgtatttttgattttttttcgggttgttgcgaggcggagtcggttcggtttttatatttcgtattttttttttttcgtaggtcgttgcgcggttttgcgtatgttgttggtagattagggt

SEQ ID NO: 2 - DNA, 19 bp, Artificial Sequence
  Primer Oligonucleotide
atttgggaaagagggaaag

SEQ ID NO: 3 - DNA, 20 bp, Artificial Sequence
  Primer Oligonucleotide
ccctaccaaacacatactcc

SEQ ID NO: 4 - DNA, 20 bp, Artificial Sequence
  Primer Oligonucleotide
gtattaggttcgggttttcg

SEQ ID NO: 5 - DNA, 22 bp, Artificial Sequence
  Primer Oligonucleotide
taataacgaaaactacgacgac

SEQ ID NO: 6 - DNA, 20 bp, Artificial Sequence
  Primer Oligonucleotide
gtattaggtttgggtttttg

SEQ ID NO: 7 - DNA, 22 bp, Artificial Sequence
  Primer Oligonucleotide
taataacaaaaactacaacaac

SEQ ID NO: 8 - DNA, 22 bp, Artificial Sequence
  Primer Oligonucleotide
gggaaagagggaaaggtttttt

SEQ ID NO: 9 - DNA, 18 bp, Artificial Sequence
  Primer Oligonucleotide
ctaaatcccctaaacccc

SEQ ID NO: 10 - DNA, 24 bp, Artificial Sequence
  Primer Oligonucleotide
agagtttttagtatggggttaatt

SEQ ID NO: 11 - DNA, 17 bp, Artificial Sequence
  Primer Oligonucleotide
ccccaatactaaatcac

SEQ ID NO: 12 - DNA, 32 bp, Artificial Sequence
  Blocker Oligonucleotide
ggttaattcgtagtattaggttcgggttttcg

SEQ ID NO: 13 - DNA, 29 bp, Artificial Sequence
  Blocker Oligonucleotide
atactaaatcacgacgccgaccgctcttc

SEQ ID NO: 14 - DNA, 32 bp, Artificial Sequence
  Blocking Oligonucleotide
ggttaatttgtagtattaggtttgggtttttg

SEQ ID NO: 15 - DNA, 29 bp, Artificial Sequence
  Blocking Oligonucleotide
atactaaatcactacaccaaccactcttc